(19) United States
(12) Patent Application Publication
(10) Pub. No.: US 2023/0103072 A1
(43) Pub. Date: Mar. 30, 2023

(54) DEEP-SCALING AND MODULAR INTERCONNECTION OF DEEP ULTRAVIOLET MICRO-SIZED EMITTERS

(71) Applicant: UNIVERSITY OF SOUTH CAROLINA, Columbia, SC (US)

(72) Inventors: RICHARD FLOYD, WEST COLUMBIA, SC (US); ASIF KHAN, IRMO, SC (US); MIKHAIL GAEVSKI, WEST COLUMBIA, SC (US); MVS CHANDRASHEKHAR, COLUMBIA, SC (US); GREGORY SIMIN, COLUMBIA, SC (US)

(21) Appl. No.: 17/747,351

(22) Filed: May 18, 2022

Related U.S. Application Data
(60) Provisional application No. 63/224,705, filed on Jul. 22, 2021.

(51) Int. Cl. H01L 33/62; H01L 33/32; H01L 27/15; H01L 33/64; H01L 33/00

(52) U.S. Cl. CPC H01L 33/62; H01L 27/156; H01L 33/32; H01L 33/0075; H01L 33/641; H01L 2933/0066

(57) ABSTRACT

A 1.8-times improved light extraction efficiency (LEE) is reported under DC test conditions for truncated cone AlGaN DUV micropixel LEDs when the pixel size was reduced from 90 to 5 µm. This is shown to be a direct consequence of the absorption of the TM-polarized photons travelling in a direction parallel to the device epitaxial layers. Presently disclosed cathodoluminescence measurements show the lateral absorption length for 275 nm DUV photons to be 15 µm, which is ~1000 times shorter than that for waveguiding in the A_{0.65}Ga_{0.35}N cladding layers. Results show the re-absorption of this laterally travelling emission by the multiple quantum wells and the p-contact GaN layer to be a key factor limiting the LEE. Hence, for DUV emitters, scaling down to sub-20 µm device dimensions is critical for maximizing LEE. Presently disclosed sub-20 µm AIGaN-based LEDs do not show pronounced edge recombination effects. The peak light output power was further increased for all the devices after the addition of a semi -reflective Al₂O₃/Al heat spreader despite the reduction in sidewall reflectivity.

### CROSS REFERENCE TO RELATED APPLICATION

This application claims filing benefit of U.S. Provisional Pat. Application Serial No. 63/224,705, having a filing date of Jul. 22, 2021, entitled “Deep-scaling and Modular Interconnection of Deep Ultraviolet Micro-Sized Emitters,” which is incorporated herein by reference.

### STATEMENT REGARDING SPONSORED RESEARCH OR DEVELOPMENT

This invention was made with government support under Grant No. W911NF18-1-0029, awarded by the Army Research Office, and under Grant No. ONR N00014-18-1-2033, awarded by the Office of Naval Research DARPA Dream. The government has certain rights in the invention.

### BACKGROUND OF THE PRESENTLY DISCLOSED SUBJECT MATTER

Group III-Nitride materials-based visible emission LEDs have emerged as a disruptive technology in the fields of lighting,¹ communications, ^{2,3,4} and displays.^{5,6} Shorter wavelength LEDs from the ultrawide bandgap (UWBG) ternary compound AlₓGa₁₋ₓN with tunable emissions in the UV spectrum (210 nm-360 nm) are now poised to displace toxic mercury-based light sources.⁷

Over the past decade aluminum gallium nitride (AlGaN) LEDs operating in the DUV spectral region λₑₘᵢₛₛᵢₒₙ < 300 nm) have been deployed in novel applications including autonomous drone-based sterilization and sanitization systems,⁸ point-of-use water purification systems,⁹ phototherapeutics,¹⁰ gas sensors,¹¹ and non-line-of-sight (NLOS) communications.¹² Nevertheless, the external quantum efficiency (EQE) of AlGaN materials-based LEDs is still significantly lower than that of their visible counterparts.¹⁰ The low EQE is rooted in the low light extraction efficiency (LEE) and thermal management issues in AlGaN-based emitters.^{13,14}

Unlike visible LEDs, the strong absorption of DUV photons by the p-GaN hole supply layer and Ni/Au contact metal stack leads to a complete loss of about half of the vertically travelling TE-polarized photons. Thus, extraction of the emitted radiation is best accomplished through the substrate side of the wafer. However, the widespread use of low cost, non-conductive sapphire substrates restricts the extraction cone to +/-22°, crippling the substrate side LEE.¹⁵

To avoid excessive absorption of the AlₓGa₁₋ₓN active region emission travelling toward the substrate, an even higher Al content transparent n-contact AlₓGa₁₋ₓN epilayer is required. The transverse magnetic/transverse electric (TM/TE) polarization ratio increases with active region Al content, leading to an increased number of in-plane photons which are quickly reabsorbed.^{16,17} Moreover, the ionization of the p- and n-dopant acceptors and donors decreases with increasing Al mole fraction, leading to current crowding and series resistance issues.¹⁸

The consumer demand for point-of-use purification and disinfection has been tremendous since the emergence of the novel coronavirus (COVID-19) and its deactivation with DUV light. AlGaN DUV LEDs are the key to these important air-water purification and germicidal applications. Currently, mercury-based sources dominate the market for systems requiring high DUV radiation doses. Their use in applications such as face mask disinfection and ventilation systems is problematic due to mercury toxicity.

The last two decades have seen intense development to improve performance of milliwatt power AlGaN DUV LEDs. Despite this, the reported external quantum efficiency (EQE) and wall plug efficiency for AlGaN DUV LEDs are well below their visible counterparts. This is primarily due to low light extraction efficiency (LEE) and thermal issues which are reduced, but not eliminated, even in flip chip LEDs. The junction heating of AlGaN DUV LEDs leads to efficiency droop, early power saturation, and reduced device lifetime. A key contribution to device self-heating is from its series resistance, which consists of contributions from the contacts, lateral spreading, and the vertical epilayer resistances. For DUV LEDs, the Ultra-Wide Bandgap (UWBG) AlGaN (3.43-6.0 eV) also dictates a high operating voltage. Although progress has been made in increasing the doping efficiency, the large ionization energy of the p-dopant acceptors results in lower free hole concentrations for UWBG AlGaN, leading to higher contact and epilayer resistances. Furthermore, the thermal conductivity of ternary AlGaN layers constituting DUV LEDs is lower than that for the binary layers of the visible LEDs.

The current spreading and series resistance issues in DUV LEDs were first addressed by using a 10 × 10 array of interconnected micropixel LEDs. That work used 25 µm diameter pixels with an interpixel gap of 15 µm where the interconnected n-ohmic contact, which blanketed the area surrounding all individual micropixels, was placed. The interconnected micropixel design increases the light output power (LOP), reduces the series resistance, increases the device reliability, and largely eliminates current crowding.

A published study of the size-dependent optothermal properties of 400 nm emission InGaN single-pixel LEDs found that the maximum power density (brightness), spectral stability, and thermal properties improve as the pixel size reduced from 300 to 20 µm. (Z. Gong, et al., J. Appl. Phys. 107, 013103 (2010)). This was due to an increased ratio of the device sidewall surface area and the mesa volume which facilitated efficient sidewall assisted out radiation of the generated heat.

A similar trend was observed for quaternary InAlGaN micro-LED arrays λₑₘᵢₛₛᵢₒₙ = 305-325 nm), where the size limit (~10 µm) was defined by an onset of saturation of the thermal resistance. (N. L. Ploch et al., IEEE Trans. Electron Devices 60, 782 (2013)). They concluded that further pixel size reduction would likely reduce the optothermal performance due to increased leakage currents at the mesa perimeters, but no studies of size-dependent LOP nor thermal impedance have been reported for AlGaN DUV micro-LEDs.

### SUMMARY OF THE PRESENTLY DISCLOSED SUBJECT MATTER

Aspects and advantages of the presently disclosed subject matter will be set forth in part in the following description, may be apparent from the description, or may be learned through practice of the presently disclosed subject matter.

Broadly speaking, the presently disclosed subject matter relates to deep-scaling and modular interconnection of deep ultraviolet (DUV) micro-sized emitters.

More particularly, certain presently disclosed subject matter relates to enhanced light extraction efficiency (LEE) of relatively smaller AlGaN DUV light-emitting diodes (LEDs).

One of the main problems with DUV LEDs is that they get hot while they are operating, limiting the amount of power that you can put in the LED, which limits how much light the LED can produce. We have greatly alleviated this thermal management issue for DUV LEDs by shrinking down the size of the devices to extremely small (micro-LED) sizes and equipping them with an effective built-in heat sink which allows them to continue to efficiently produce light as the input power is cranked up. Our presently disclosed devices are able to produce significantly more light for its size because the heat it generates while turned on is whisked away from the micro-LED by the built-in heat sink. We explored how to connect these micro-LEDs together to scale our technology up and make big LEDs, which can produce a larger amount of light overall compared to our very small micro-LED.

We developed a new way to connect multiple micro-LED together that is highly scalable using a modular approach. First, we made groups of micro-LEDs by connecting multiple micro-LEDs that are in close proximity to each other with a metal heat sink; then, we connected all these groups of micro-LEDs together to make an LED of arbitrary size. With this modular approach for scaling, we can ensure an even distribution of the input power among the individual micro-LEDs for all the groups while keeping the boost to thermal performance that the individual micro-LEDs offer.

The presently disclosed subject matter may be useful in a number of different settings. For example, these may include germicidal and virus killing; water purification (large scale and point-of-use); sterilization of surfaces (e.g., refrigerators, drinking glasses at bars and restaurants, etc.); deep ultraviolet optical communications; polymer curing; sterilization of food; and microscale light emission source and/or detector for DUV photonics integrated circuits. Accordingly, potential uses may relate to the food packaging industry, point-of-use water purifiers, large scale water purification, air purification, surface and object sterilization, germ killing and viral deactivation, and curing applications (such as UV-sensitive polymers).

Some potential advantages may relate to greatly improving device thermal management over current devices; reducing light absorption from n-contact metals compared to current art micropixel technology; increased external quantum efficiency over current devices; increased peak output power from increased efficiency and improved thermal management compared to current devices; increased brightness (W/cm²) compared to current devices; and more compatible sizing for optical tweezing, multi-sample chemical analysis, and display-based applications, along with expected ~30% decrease in dollar-per-watt cost of DUV lighting compared to current devices using the presently disclosed modular interconnection scheme.

Previously, our group has shown that compared to large area LEDs, interconnected DUV micropixel LEDs (micro-LEDs) can reduce the device series resistance, largely alleviate current crowding, improve reliability, and increase the maximum light output power (LOP) via a reduction of the thermal resistance.^{19,20,21}

We presently disclose results of systematic study of individual and interconnected AlGaN MQW micropixel DUV LEDs with pixel sizes from 5 to 15 µm. We also explore a new interconnected micropixel design, which enabled high brightness and high power DUV emission. For this new design, the blanket n-contact network between the individual micropixels was removed to increase the active area coverage and reduce the optical absorption. The n-contact for this present work forms a narrow picture frame border around a densely packed subarray of interconnected micropixels. The subarray interconnection process also passivates the pixel sidewalls and spreads the self-generated heat away from the individual micropixels while avoiding current crowding. Then, multiple subarrays are interconnected as shown in FIG. 1A. The completed device is suitable for subsequent electroplating and flip chip packaging. All the micropixel arrays of this study with different micropixel diameters have a total junction area of 6.36 × 10⁻⁵ cm², which is also the same as that of a reference, 90 µm diameter standalone LED.

Individual and interconnected AlGaN (semiconductor material) multiple quantum well (MQW) micropixel DUV LEDs with pixel sizes from 5 to 15 µm and a presently disclosed interconnected micropixel design enables high brightness and high power DUV emission with area scalability. For this presently disclosed design, the blanket n-contact network between the individual micropixels is removed to increase the active area coverage and reduce the optical absorption. The n-contact for this presently disclosed state-of-the-art design forms a narrow picture frame border around a densely packed subarray of interconnected micropixels. The presently disclosed subarray interconnection process also passivates the pixel sidewalls and spreads the self-generated heat away from the individual micropixels while avoiding current crowding. Then, multiple subarrays are interconnected. This completed device is suitable for subsequent electroplating and flip chip packaging. The reduction in pixel size down to 5 µm was shown to greatly reduce the thermal impedance of a micropixel array compared to a broad mesa device. This is primarily from the reduction in device series resistance, a division of the input through an increased number of micropixels, and an increased sidewall out radiation of the self-generated heat with decreasing pixel size. Due to the 3.75 × reduction in thermal impedance compared to the 90 µm diameter reference LED, the highest on wafer output powers (15.25 × higher than that of an equal junction area 90 µm diameter reference LED) was delivered by an interconnected array of 5 µm diameter micropixels. These are the smallest and the brightest reported DUV micropixel LEDs to date.

The presently disclosed device fabrication procedure includes first using etching to define the micropixels and access the n-contact making layer. The resulting structure can have either vertical or inclined sidewalls or a combination of the two. Annealing in a nitrogen environment was then performed to activate the p-dopants. Then, a narrow picture frame n-contact was fabricated around single-pixels (for standalone devices) and the subarrays of pixels (for interconnected devices), although one could leave sections of this frame open. Truly, it need not even be a continuous path of metal provided that it is eventually connected to a common supply terminal. A noncontinuous metal, which is not later made continuous, is also possible to form arrays with independent electronic control of either individual micropixels or the subarrays of micropixels.

The n-contact metal stack was deposited via e-beam and annealed in forming gas by rapid thermal annealing (RTA). The internal dimension of this n-contact border was for all cases < 100 µm, which precludes electrical current crowding. Though, with further improvement to the n-AlGaN sheet resistance or a denser packing of the pixels, subarrays with an increased number of micropixels are achievable.

Following the n-contact, p-contacts were formed for the individual micropixels and annealed on a hotplate in an O₂ environment.

The first micropixel interconnection stage began with a conformal electrical current isolating film. Windows above the p-contacts were then opened by dry etching, though wet chemical etching is also possible. This was followed with electron beam deposition of a thick reflective aluminum heat-spreader to interconnect the individual micropixels, thereby forming the subarrays. The Al interconnect blanketed the entire internal area of the n-ohmic picture frame borders, although different amounts and styles of coverage are possible. The second stage of interconnection started with deposition of a current-isolating layer followed by an etching step to open windows for each of the subarrays. These subarrays of micropixels were then interconnected to form LEDs. The final metal stack deposition blanketed and interconnected the arrays of subarrays, although different amounts of coverage are also possible.

One presently disclosed exemplary embodiment relates to a LED, preferably comprising an AIGaN-based micropixel LED device having a pixel p-contact diameter size of 20 µm or less and operating in the DUV spectral region having wavelength emissions of less than 300 nm.

Another presently disclosed exemplary embodiment relates to a modular LED array. Such array preferably comprises a plurality of respective aluminum gallium nitride (AlGaN) multiple quantum well (MQW) micropixel LEDs operating in the DUV spectral region with λₑₘᵢₛₛᵢₒₙ < 300 nm. Further, such plurality of AlGaN MQW DUV LEDs preferably is respectively arranged in an array interconnected by a metal heat sink and connected to a common supply terminal. Yet further, preferably such LEDs have respective pixel sizes from 5 to 20 µm in diameter and respectively have an added heat sink layer.

It is to be understood from the complete disclosure herewith that the presently disclosed subject matter equally relates to both apparatus and corresponding and/or related methodology. One presently disclosed exemplary methodology preferably relates to methodology for forming a LED modular device, comprising fabricating an AlGaN-based micropixel LED device operable in the DUV spectral region as to have a pixel diameter size of 20 µm or less.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding and/or related methodologies as associated with practice of any of the present exemplary devices, and vice versa.

### BRIEF DESCRIPTION OF THE FIGURES

A full and enabling disclosure of the presently disclosed subject matter, including the best mode thereof, directed to one of ordinary skill in the art, is set forth in the specification, which makes reference to the appended Figures, in which:

FIG. 1A illustrates multiple schematic images of presently disclosed exemplary device layouts, and represents an interconnection process overview with micrographs at each level of fabrication, including representations of single micropixels, subarrays of micropixels, arrays of subarrays, subarray formation, and fully fabricated devices (such as a lamp or lighting system);

FIG. 1B illustrates a cross-sectional schematic of structural details for exemplary embodiment of presently disclosed DUV LEDs;

FIG. 1C illustrates a scanning electron microscope (SEM) image of an exemplary embodiment of a single 5 µm pixel (defined by the p-contact diameter) with the Al heat-spreader;

FIG. 1D is a Table which summarizes details for various device geometries schematically shown in FIG. 1A and includes relevant parameters for presently disclosed devices (with S.S.A/V the ratio of sidewall surface area to the mesa volume);

FIG. 2A illustrates a graph of the measured electroluminescence spectra of a single presently disclosed 5 µm pixel with the on-wafer Al heat-spreader under various CW pump currents;

FIG. 2B illustrates a graph of the junction area normalized I-L characteristics for presently disclosed single micropixels against a reference LED under CW pump and a graph showing the J-V characteristics for the same;

FIG. 3A illustrates a graph of I-V characteristics for the presently disclosed parallel connected micropixel arrays and a reference LED (with all of the devices having identical junction areas);

FIG. 3B illustrates a graph of absolute I-L under CW pump for equal junction area LEDs and a graph showing the pulsed mode output power for the same and the pulsing conditions; the other image is of a 6 × 6 subarray of 5 µm pixels and shows a 3 × 3 array comprised of such 6 × 6 subarrays of 5 µm pixels under CW pumping for which over 95% of the pixels shown in the completed 3 × 3 array are operating;

FIG. 3C is a Table showing the maximum brightness of several reported AlGaN DUV LEDs including flip chip, tunnel junction (TJ), nanopatterned sapphire substrates (NPSS), and state-of-the-art flip chip multi-die encapsulated devices (where SS denotes sapphire side light extraction and TS denotes topside (p-electrode));

FIG. 4 illustrates a graph of measured junction temperature rise as a function of CW input power for the equal junction area devices, with a linear fit used to extract the thermal impedances, and a graph showing the linear relationship between measured thermal impedance and pixel size for all the equal junction area devices;

FIG. 5 illustrates multiple SEM images of sidewall profiles for exemplary embodiments of presently disclosed devices;

FIG. 6 illustrates cathodoluminescence (CL) imaging of a truncated cone 90 µm micropixel with the monochromatic intensity plot overlaid and with the CL signal smoothed by a 10-point average and then fitted with an exponential curve to extract the lateral absorption length of presently disclosed device epistructures;

FIG. 7 illustrates a graph of electroluminescence emission spectra for presently disclosed device subject matter at various pump currents;

FIG. 8A illustrates a graph of DC pump I-V-L characteristics for bare sidewall devices with vertical sidewalls;

FIG. 8B illustrates a graph of DC pump I-V-L characteristics for bare sidewall devices with slanted sidewalls;

FIG. 9 illustrates a graph of external quantum efficiency (EQE) for bare sidewall devices;

FIG. 10 illustrates a graph representing light extraction efficiency (LEE) enhancement over vertical sidewall devices as a function of the mesa radius;

FIG. 11A illustrates a graph of DC pump I-V-L characteristics for devices equipped with the Al₂O₃/Al heat-spreader having vertical sidewalls;

FIG. 11B illustrates a graph of DC pump I-V-L characteristics for devices equipped with the Al₂O₃/Al heat-spreader having B slanted sidewalls;

FIG. 12A is a graph of pulsed mode I-L characteristics for devices equipped with the Al₂O₃/Al heat-spreader having vertical sidewalls; and

FIG. 12B is a graph of pulsed mode I-L characteristics for devices equipped with the Al₂O₃/Al heat-spreader having B slanted sidewalls.

Repeat use of reference characters in the present specification and figures intended to represent the same or analogous features or elements or steps of the presently disclosed subject matter.

### DETAILED DESCRIPTION OF THE PRESENTLY DISCLOSED SUBJECT MATTER

It is to be understood by one of ordinary skill in the art that the present disclosure is a description of exemplary embodiments only and is not intended as limiting the broader aspects of the disclosed subject matter. Each example is provided by way of explanation of the presently disclosed subject matter, not limitation of the presently disclosed subject matter. In fact, it will be apparent to those skilled in the art that various modifications and variations can be made in the presently disclosed subject matter without departing from the scope or spirit of the presently disclosed subject matter. For instance, features illustrated or described as part of one embodiment can be used with another embodiment to yield a still further embodiment. Thus, it is intended that the presently disclosed subject matter covers such modifications and variations as come within the scope of the appended claims and their equivalents.

The present disclosure generally relates to deep-scaling and modular interconnection of deep ultraviolet (DUV) micro-sized emitters and to individual devices for use in such configurations.

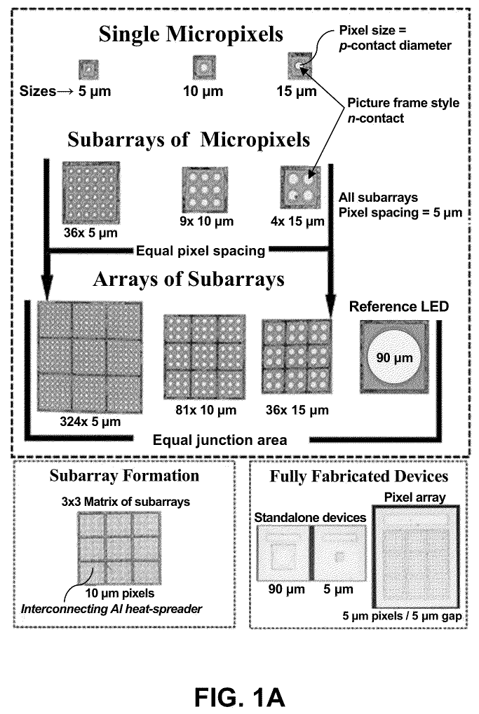

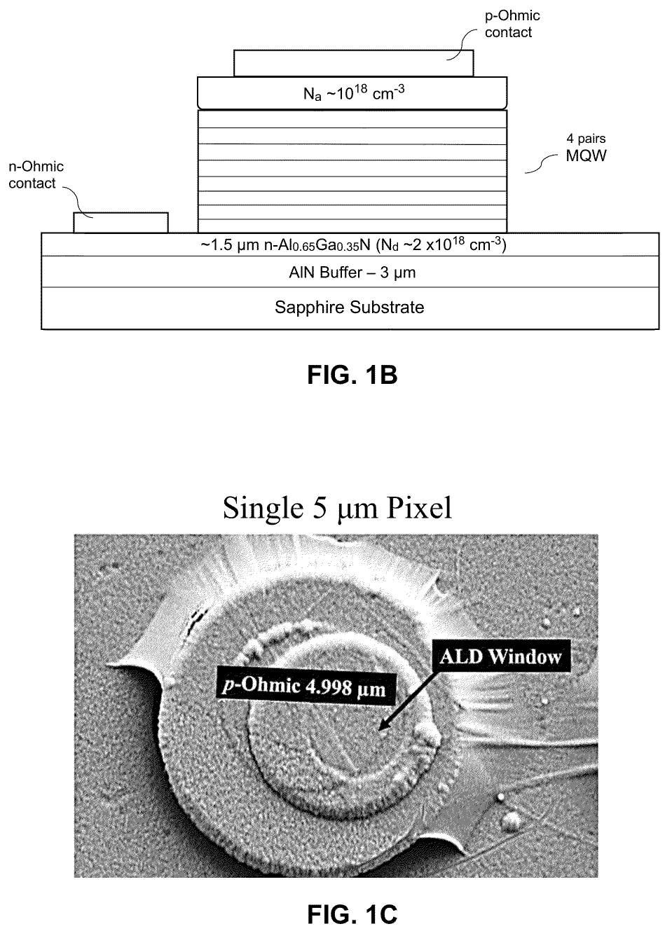

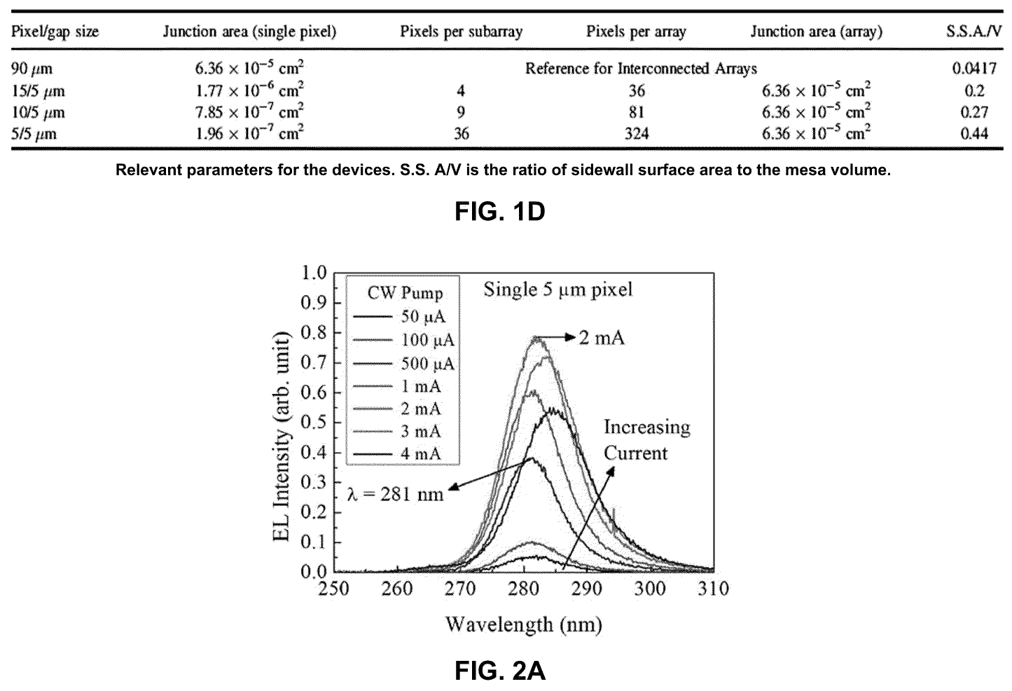

More specifically, we presently disclose results of systematic study of individual and interconnected AlGaN MQW micropixel DUV LEDs with pixel sizes from 5 to 15 µm. We also explore a new interconnected micropixel design, which enables high brightness and high power DUV emission. For this new design, the blanket n-contact network between the individual micropixels is removed to increase the active area coverage and reduce the optical absorption. The n-contact for this present work forms a narrow picture frame border around a densely packed subarray of interconnected micropixels. The subarray interconnection process also passivates the pixel sidewalls and spreads the self-generated heat away from the individual micropixels while avoiding current crowding. Then, multiple subarrays are interconnected as shown in FIG. 1A. The completed device is suitable for subsequent electroplating and flip chip packaging. All the micropixel arrays of this study with different micropixel diameters have a total junction area of 6.36 × 10⁻⁵ cm², which is also the same as that of a reference, 90 µm diameter standalone LED.

The epilayer structure was grown over 3 µm-thick thermally conductive AlN templates over c-plane sapphire substrates using metalorganic chemical vapor deposition It consists of an MOCVD-grown AlN (~3.5 µm)/basal plane sapphire template with a 1.5 µm thick n⁺-Al_{0.65}Ga_{0.45}N n-contact/cladding layer (N_{d} ~ 2 × 10¹⁸ cm⁻³) and is followed by 4 pairs of Al_{0.6}Ga_{0.4}N/Al_{0.35}Ga_{0.65}N multiple quantum wells λₑₘᵢₛₛᵢₒₙ ~ 280 nm) and an electron blocking AlGaN, a polarization doped graded composition p⁻AlGaN, and a Mg-doped hole-injection p⁺-GaN cap layer. The device structure and the epilayer growth details are shown in FIG. 1B. The device fabrication procedure consisted of first using a Cl₂/Ar chemistry inductively coupled plasma reactive ion etching (ICP-RIE) to define the micropixels and to access the n-contact making n⁺-Al_{0.65}Ga_{0.35}N layer. Annealing in a nitrogen environment was then performed at 750° C. to activate the Mg dopants. Then, a narrow picture frame n-contact (5 µm wide) was fabricated around single-pixels (for standalone devices) and the subarrays of pixels (for interconnected devices). The n-contact metal stack Zr(150 Å)/Al(1200 Å)/Mo(350 Å)/Au (500 Å) was deposited via e-beam and annealed at 950° C. for 3 minutes in forming gas by rapid thermal annealing (RTA). The internal dimension of this n-contact border was for all cases < 100 µm. Prior work indicates that this geometry precludes current crowding. From the n-contact TLM measurements, the sheet resistance for the epilayer structure and the contact resistance were Rₛₕ=120 Ω/Y and ρ_{c} = 6 × 10⁻⁴ Ω▪cm². Following the n-contact, Ni/Au p-contacts were formed over the individual micropixels and annealed at 500° C. for 5 minutes on a hot plate in an O₂ environment. The p-metal dimensions were 5, 10 and 15 µm diameter for the micropixels.

The first micropixel interconnection stage began with atomic layer deposition (ALD) of a conformal 75 nm-thick Al₂O₃ film. Windows above the p-contact regions of the individual micropixels were then opened by ICP-RIE with a high power Cl₂/BCl₃/Ar-based etch. This was followed with photoresist masking and electron beam deposition of a 300 nm-thick reflective aluminum heat-spreader to interconnect the individual micropixels, thereby forming the subarrays. The Al interconnect blanketed the entire internal area of the n-ohmic picture frame borders. An SEM image of a fabricated micropixel with a p-ohmic diameter of 5 µm and the Al heat-spreader is shown in FIG. 1C.

The second stage of interconnection started with plasma-enhanced chemical vapor deposited SiO₂ (400 nm), followed by a SF₆/CF₃H/Ar dry etching with RIE to open windows for each of the subarrays. For each mesa diameter, nine subarrays (of micropixels) were then interconnected to form LEDs with the same emission area as the reference 90 µm diameter single-pixel LED. The final metal stack deposition blanketed and interconnected the 3 × 3 arrays of subarrays. FIG. 1D summarizes details for the various device geometries schematically shown in FIG. 1A.

Both standalone micropixels and the 3 × 3 arrays of interconnected micropixel subarrays were then measured and compared to the reference LED for their current-voltage-light output (I-V-L) and external quantum efficiency (EQE). An Si-photodiode and a calibrated photometer were used for the measurements. Using a thermal-driven spectral shift approach, the junction temperature versus input electrical power was measured for the micropixel arrays and the reference LED.

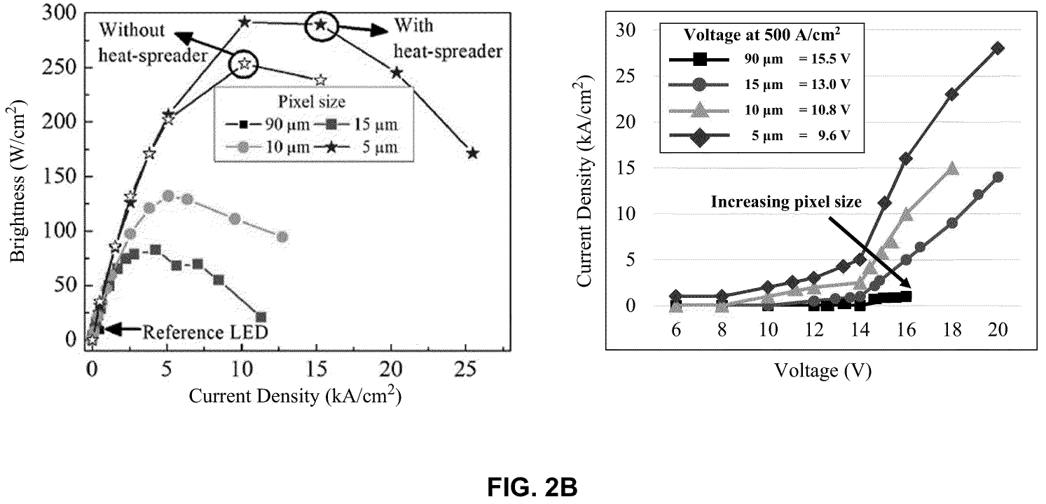

All the measurements were made on-wafer. The pulsed measurements for the micropixel arrays and the reference LED were conducted using 500 ns wide pulses at 0.05% duty cycle to minimize device heating. FIG. 2A shows the electroluminescence (EL) spectra of a single 5 µm pixel with the Al heat-spreader. The EL emission obtained at 2 mA (10.2 kA cm⁻²) under continuous wave (CW) pumping has undergone a small redshift, indicating moderate device self-heating, which becomes more severe with increasing injection current. The junction area normalized I-V-L characteristics for the single-pixel devices under CW pump are plotted in FIG. 2B.

The light generation increases with pump current until junction heating leads to efficiency droop. The brightness peaked at 291 W cm⁻² at 10.2 kA cm⁻² for the single 5 µm pixel with the Al heat-spreader. This was nearly a factor of 30 higher than the reference LED. As the pixel size shrinks, less absolute injection current (and total input power) is required to reach the same current density. Despite the increasing series resistance with decreasing pixel size for individual micropixels arising from the reduced conductive cross-sectional area of the epistructure and the ohmic contacts, the total joule heating for a given current density decreases with decreasing pixel size enabling high current density operation.

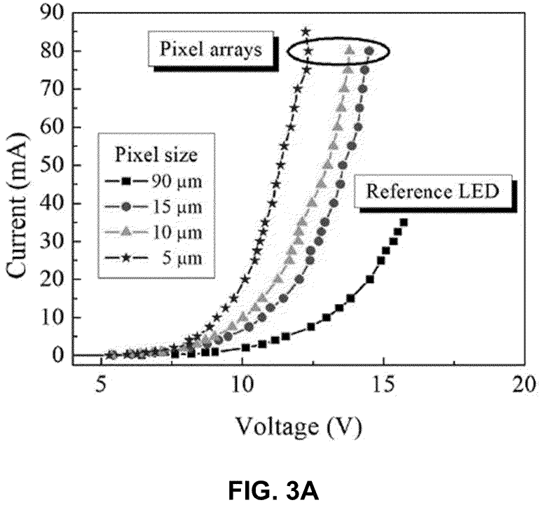

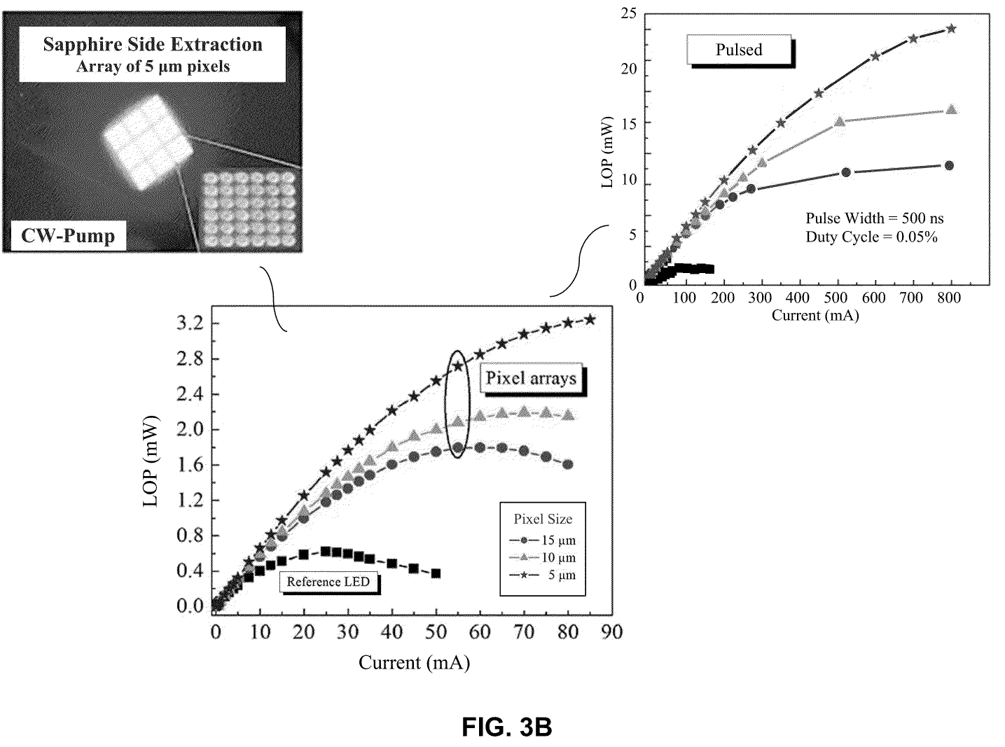

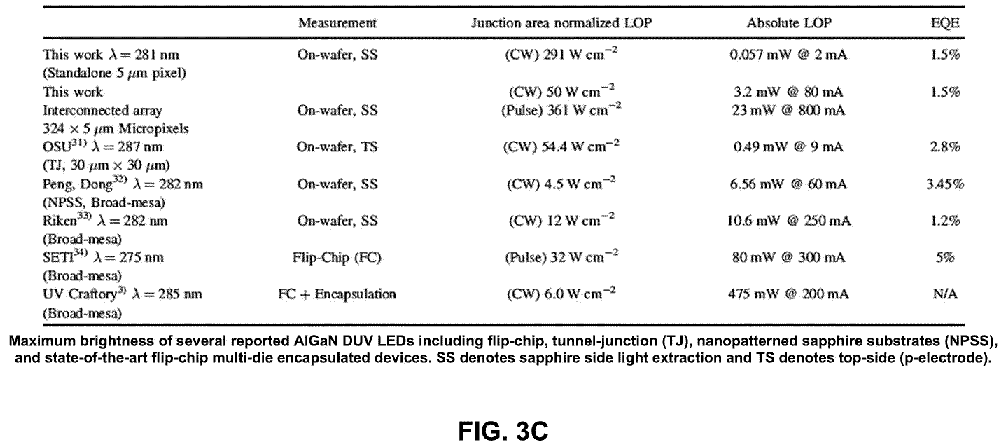

In FIG. 3A, the I-V characteristics of the equal junction area micropixel arrays and reference LED are plotted. The operating voltage and series resistance for the micropixel arrays is less than that of the broad mesa reference LED and decreases with decreasing pixel size due to the growing area of the n-contact with the increasing chip footprint required to make equal junction area devices. The junction area normalized brightness at low input powers was found to be identical for a single 5 µm pixel (without the Al heat-spreader) and an interconnected array of the same size micropixels. This indicates minimal optical loss from the interconnection process. From the I-L data of FIG. 3B, the highest output powers (and brightness) of 3.2 mW (50 W cm⁻²) and 23 mW (361 W cm⁻²) were delivered by the interconnected array of 5 µm pixels under CW and pulsed pumping respectively. This translates to a 5.25-fold (CW) and 15.2-fold (pulsed) increase in maximum LOP compared to the reference LED. The bare chip peak EQE of ~1.5% was extracted from the CW data of FIG. 3B. Regardless of pixel size, a 13.5% increase in the peak EQE was measured for the micropixel arrays over the reference device. This indicates no impact from sidewall defects or leakage currents, even for pixel sizes as small as 5 µm, which may be attributed to the post-mesa formation annealing.

Our results suggest that, unlike GaN/InGaN LEDs, the ideal mesa size for optimal performance of AlGaN DUV micro-LEDs resides in the sub-10 µm regime. They also support the assertion that the substantially higher peak LOP over the reference LED was enabled by improved thermal management of the micropixel arrays. Our interconnected micropixel design in this study is therefore an attractive approach to overcome thermal droop, a critical limitation for high LOP in AlGaN DUV LEDs. FIG. 3C compares the peak brightness, LOP, and EQE for several reported research and commercial DUV LEDs.

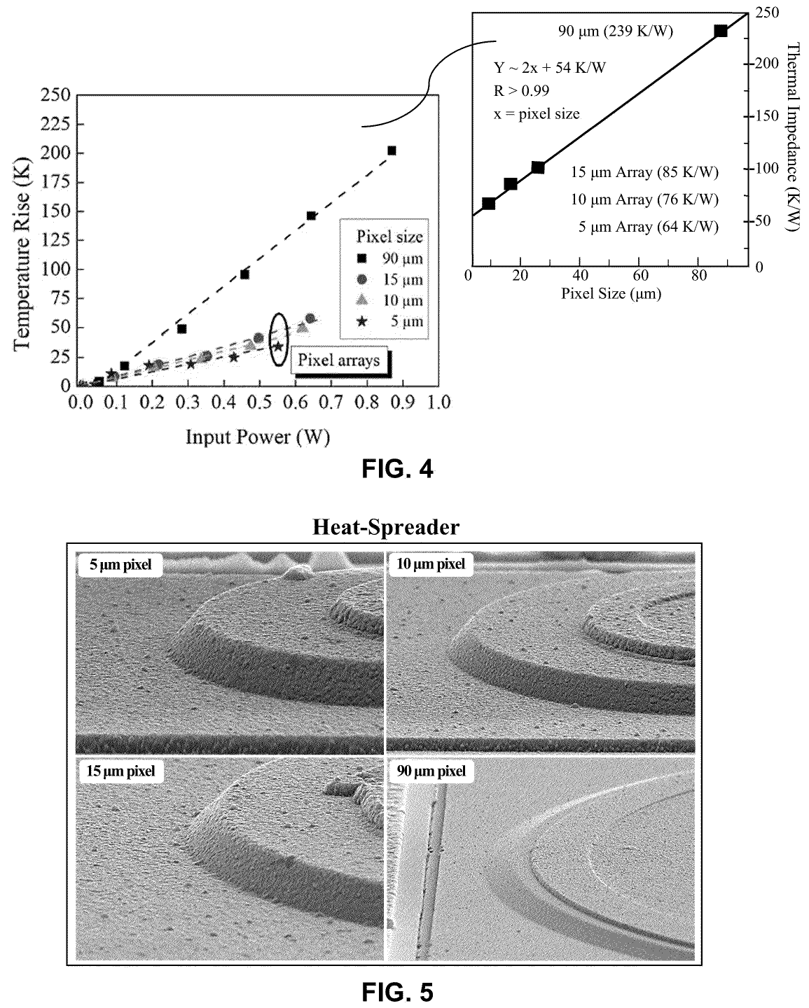

We next measured the junction temperature rise as a function of CW input power for the micropixel arrays and for the reference LED using the well-established electroluminescence spectral shift method (see FIG. 4).

Two sets of calibration measurements were carried out before device temperature quantification: (1) measurement of the redshift of the emission spectra with increasing junction temperature using a heated stage at a fixed pulsed pump current; and (2) measurement of the blueshift of the emission spectra at room temperature with increasing pulsed pump current. Both measurements were made using current pulses with a duration of 500 ns, a duty cycle of 0.05%, and a rest time of 10 minutes (between data points) to avoid pump current induced device self-heating. The maximum redshift was 2.58 nm for a junction temperature range of 298-423 K. The largest observed blueshift of 0.782 nm was from an interconnected array of 5 µm pixels at an injection current of 50 mA. The mechanisms underlying the blueshift have been reported by multiple groups across several III-nitride platforms.

After the calibrations were performed, the device emission spectra were measured with increasing CW pump current in a room temperature environment to estimate the junction temperature rise with input power. Then, for each pixel size, the spectral contribution of the current-dependent blueshift was subtracted from the junction temperature rise spectral data to remove its influence on the measurement. A linear fit was applied to the measured data in FIG. 4 to extract the thermal impedances.

The steeper slope for the reference device, compared to those of the interconnected micropixel arrays, indicates significantly higher joule heating. The reduction in thermal impedance for the interconnected micropixel LED consisting of 5 µm pixels compared to the reference device was approximately 3.75-fold, supporting the origin of the substantially increased peak LOP to be thermal rather than optical. The linear fit in the inset underscores the strong dependence of thermal impedance on pixel size arising from the distribution of the input current through an increased number of micropixels and the increased sidewall out radiation of self-generated heat. The inset suggests that further reduction of pixel size is unlikely to significantly improve the on-wafer thermal performance.

In summary, we have presented a new design for the interconnected DUV micro-LED to enable densely packed scalable arrays of sub-20 µm diameter micropixels. We studied the light output and thermal properties of the devices and compared them to a reference LED with identical junction area. The reduction in pixel size down to 5 µm was shown to greatly reduce the thermal impedance of a micropixel array compared to a broad mesa device. This is primarily from the reduction in device series resistance, a division of the input through an increased number of micropixels, and an increased sidewall out radiation of the self-generated heat with decreasing pixel size. Due to the 3.75 × reduction in thermal impedance compared to the reference LED, the highest on-wafer output powers exceeding 360 W cm⁻² were delivered by an interconnected array of 5 µm diameter micropixels.

Information is presented herein on light output power and thermal impedance of 281 nm emission AlGaN based micropixel LEDs. A modular interconnected micropixel array design enables dense packing with area and power scalability. Information is shown on 5-15 µm diameter standalone devices and parallel connected micropixel arrays with 5 µm interpixel gaps. A standalone 5 µm pixel emits 291 W cm⁻² at 10.2 kA cm⁻² DC drive. A power as high as 23 mW (361 W cm⁻²) was measured at a pulsed pump current of 800 mA (~15 kA cm⁻²) for an interconnected array. These are the smallest and brightest DUV micropixel LEDs to date.

We also demonstrated a high-density dot matrix 280 nm emission micro-LED display with a pixel size of ~25 µm²² with independent control of the pixels, a requirement for display-based applications such as direct-write lithography. That same year, we demonstrated Fresnel micro-lenses directly formed on the sapphire side of micro-LED wafers, better facilitating their integration in optical systems.²³ Recently, micro-size DUV emitters were surveyed for use in optical wireless communications (OWC) and data transfer links.²⁴ To date, the highest reported modulation bandwidth for a DUV LED is 570 MHz, enabled by a single 20 µm diameter AlGaN micro-LED with a peak LOP of 130 µW.²⁵ Despite the reduced emission area for micro-LEDs, the brightness (W cm²) is remarkably enhanced, owed to their efficient light generation at kA cm² class current densities enabled by a superior removal of the self-generated heat from the device active region.¹⁹ At these levels of injection current density, the dynamic carrier lifetimes, which chiefly dictate the maximum modulation bandwidth for micro-LEDs, is significantly reduced.^{26,27} Hence, increasing the LEE and the peak brightness for DUV micro-LEDs is of particular benefit for high-bandwidth optical systems.

One powerful technique to increase the LEE of DUV LEDs is by slanting the mesa sidewalls to efficiently re-direct the in-plane TM-polarized photons toward the substrate for extraction.²⁸ Since the first report of this truncated cone architecture for DUV devices, there have been several studies on the optimization of the sidewall angle,²⁹ sidewall reflector,³⁰ and the passivating dielectric.³¹ However, there are no reports hitherto on the device size dependence of the LEE enhancement of DUV micro-LEDs. In this present disclosure, we offer the findings of such a study. Importantly, for slanted sidewall devices, the LEE enhancement in the absence of current crowding is proportional to R ▪e^{-αx}, where R is the sidewall reflectivity, α is the absorption coefficient for sideways travelling photons, and x is the lateral travel distance from the center of the mesa to the perimeter.¹⁶ Hence, we also explore effect of a semi-reflective Al₂O₃/Al heat-spreader on the device performance. The micro-LEDs used for this investigation were of sizes 5, 10, 15 and 90 µm, which are referred to as pixel sizes and are defined by the p-contact diameter. Devices with vertical and slanted sidewalls were fabricated on the same 2" wafer and possess well-matched current-voltage (I-V) characteristics.

The following portion of the presently disclosed subject matter relates to growth and fabrication of the subject exemplary structures.

Similar to our previous report,¹⁹ the epistructure for our devices consists of a double-sided polished sapphire substrate, a low-defect density 3 µm-thick AlN buffer layer,³² a 2.5 µm-thick n⁺-Al₀.₆₅Ga_{0.35}N (N_{d} ~ 5 x 10¹⁸ cm⁻³) n-contacting layer followed by a 4-pair AlGaN-based MQW active region, a 20 nm p-Al_{0.7}Ga_{0.3}N electron block layer, a 55 nm polarization-doped reverse graded p-AlₓGa₁₋ₓN (x=0.7→0.3) layer, and a 150 nm p⁺-GaN hole supply (Nₐ ~ 2 x 10¹⁸ cm⁻³) cap layer.

For the devices with slanted sidewalls, the mesa photoresist (PR) pillars were first shaped into hemispherical domes by exposing the developed PR pattern to UVA irradiation, which lowers the melting point and improves the temperature stability of the mask. This was followed by time dependent thermal reflow. Then, mesa etching was performed for both slanted and vertical sidewall devices using Cl₂/Ar chemistry by inductively coupled plasma reactive ion etching (ICP-RIE). Further details of the fabrication procedure are elsewhere.¹⁹

The scanning electron micrographs (SEM) of FIG. 5 show the slanted sidewall profiles of the co-fabricated micropixel LEDs after the device fabrication is complete. The sidewall angles were 25°, 45°, 48°, and 48° for the 90, 15, 10, and 5 µm pixels, respectively. The specific contact and sheet resistances were 1.64 x 10⁻⁴ Ω▪cm² and 80 Ω/Y for the n-side and 3.86 x 10⁻⁴ Ω▪cm², 91 k Ω/Y for the p-side.

During the development of our slanted sidewall process for sub-20 µm AlGaN devices, we found that the volume of photoresist (covering a single mesa) greatly impacted the thermal dose required to achieve the desired sidewall profile. Consequently, there are differences in the sidewall angle between the co-fabricated sub-20 µm and the 90 µm micropixels. However, varying the sidewall angle from 28°-40° was reported to have a small effect (< 0.1%) on the overall device EQE.²⁹ Hence, we do not expect the differences in sidewall angle to significantly alter the results of this study.

The following portion of the presently disclosed subject matter relates to the results and related discussion concerning the subject exemplary structures.

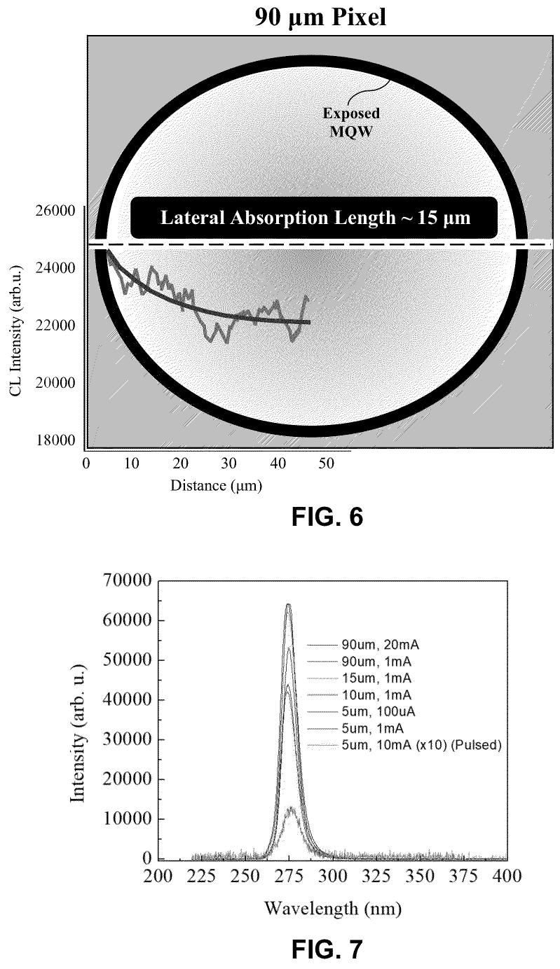

Similar to the photoluminescence (PL) technique reported for an AlGaN laser diode,³³ we used a JEOL SEM with a UV-enhanced GATAN MonoCL-2™ cathodoluminescence (CL) system and a DigiScan™ beam control unit to perform a line scan across the mesa of a 90 µm pixel with a slanted sidewall profile before metallization (see FIG. 6). The monochromatic λ_{detection} ~275 nm) CL signal was smoothed with a 10-point average and then fitted with an exponential curve to extract the absorption coefficient in the units of 1/pixel. By mapping the number of pixels covered by the CL line scan to the SEM-measured mesa diameter, the lateral absorption length within the mesa structure was estimated to be ~15 µm, which is similar to the previously established value of ~10 µm for a MQW-based LED from Monte Carlo simulation. ^{13,34} Considering the measured lateral absorption length of only 15 µm, a sub-20 µm lateral travel distance for DUV photons is critical for improving the LEE of AlGaN MQW-based LEDs.

To validate this assertion, current-voltage-output power (I-V-L) and EQE measurements were made on micropixels with bare sidewalls (after metallization) to study the size-dependent effects. Measurements were repeated after the device was equipped with a semi-reflective Al₂O₃/Al heat-spreader to study the tradeoff between the thermal enhancement of the devices (which improves light generation for micro-LEDs,¹⁹) and the reduction of sidewall reflectivity from depositing a conformal metal reflector on the dry-etched sidewalls.³⁰ All measurements were made on-wafer using a calibrated photometer and a UV-sensitized Si photodiode. The electroluminescence (EL) emission spectrum in FIG. 7 was collected using a fiber-coupled HORIBA® monochromator with a LN₂-cooled CCD array.

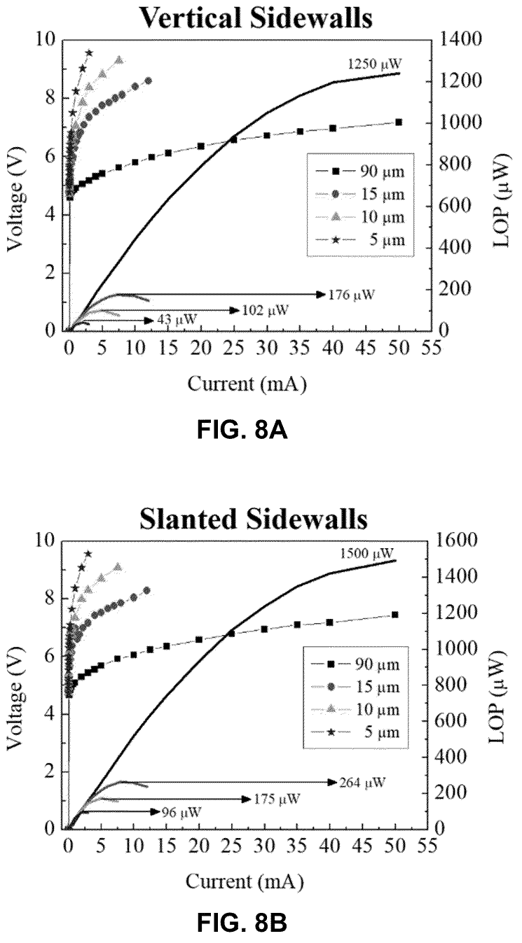

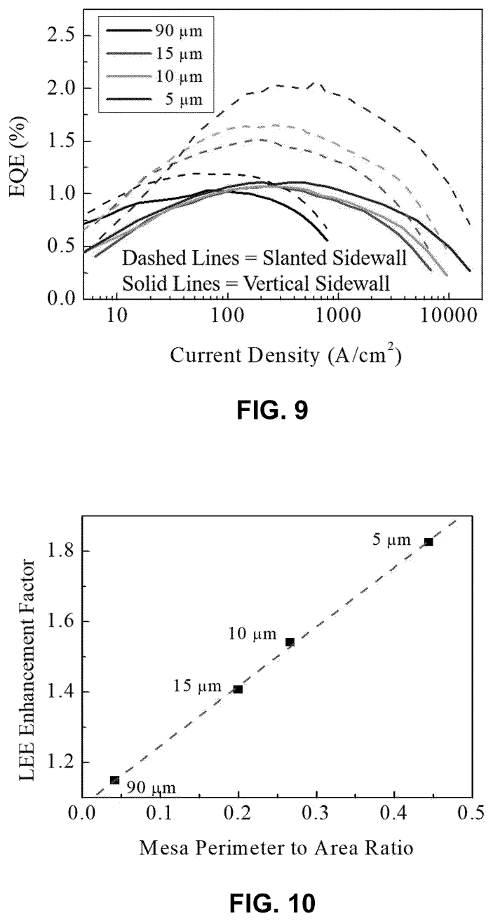

FIG. 8 shows the I-V-L curves for the vertical and slanted sidewalled micro-LEDs under DC current injection, demonstrating increased LOP and well-matched electrical characteristics for micropixels of the same size. Notably, our fabrication method precludes the voltage penalty of previous reports where the sidewall profiles were defined during ICP-RIE.^{29,30} Because the EQE (FIG. 9) extracted from FIG. 8 is higher in the case of the sub-20 µm vertical walled devices, we attribute the elevated current density at which the peak EQE occurs to an improvement of the device thermal management rather than increased edge leakage of the current.¹⁹ Moreover, the position of the peak EQE is the same for both the vertical and slanted sidewall devices of sub-20 µm dimensions, which indicates a lack of plasma-induced material damage at the device periphery,²⁹ although a pronounced size-dependent enhancement of the EQE is seen in FIG. 9 for the devices with slanted sidewalls. Using the ABCD method,³⁵ we determined that the peak internal quantum efficiency (IQE) of ~70% for our devices was independent of the pixel size and the sidewall profile. Considering the well-matched electrothermal characteristics and size independent IQE, we thus attributed the EQE enhancement to an improvement in the LEE. FIG. 10 clearly shows a strong (1/r) dependence of the LEE enhancement factor on the mesa radius. It is well established that TM-polarized emission propagates in-plane (laterally) from the point of generation, whereas TE-polarized emission travels mostly in the vertical direction.^{13,16,28} This implies that the marked LEE enhancement is predominantly from an increase in the out-coupling of TM-polarized emission. In direct agreement with the CL measurement, the EL results show that for traditional geometry broad area devices, only the in-plane (TM-polarized) emission, which is generated within a few absorption lengths of the mesa perimeter, can be extracted. This is exacerbated as λₑₘᵢₛₛᵢₒₙ is shortened and the TE/TM emission ratio shrinks. Thus, the micro-LED platform and truncated cone architecture provides an attractive route for improving the LEE as the MQW Al content is increased.

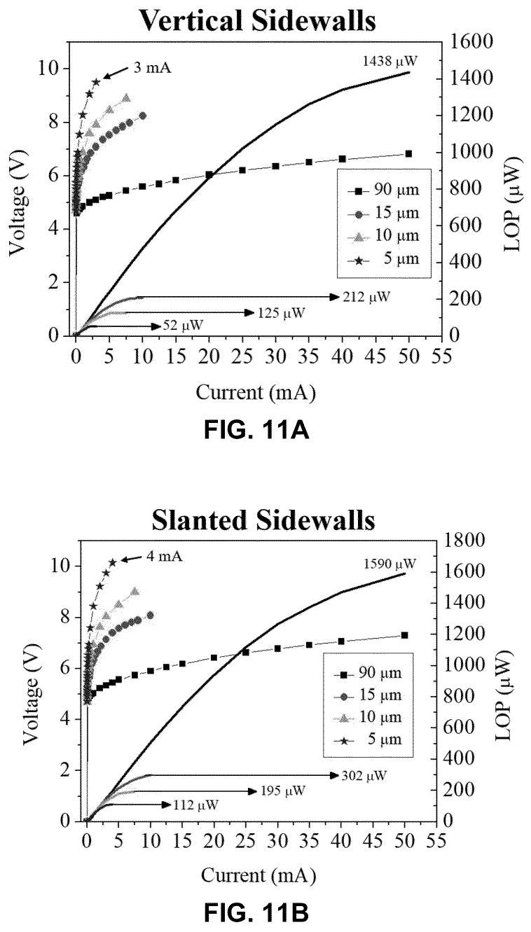

FIG. 11 shows the DC I-V-L characteristics of the devices with a semi-reflective Al₂O₃/Al heat-spreader. The I-V characteristics of the devices were unchanged, although the maximum LOP increased and thermal droop noticeably lessened compared to the bare sidewall condition. Interestingly, the onset of LOP saturation is much softer for the sub-20 µm micropixels with slanted sidewalls compared to those with vertical sidewalls, indicating improved thermal management. This arises from the highly conformal coverage of the heat-spreader in the case of the slanted sidewall devices, which better transfers the self-generated heat away from the mesa pillars, as opposed to the air-gapped sidewall contact profile formed on our vertical walled devices.¹⁹ Comparing the EQEs obtained from the data of FIG. 11 with that of FIG. 9, thermal droop also lessened at high injection current densities. Like our previous report, the EQE of the vertical sidewall devices increased by 1.15 × after the addition of the heat-spreader.¹⁹ The peak EQEs were similar for both the vertical and slanted sidewall devices after the addition of the Al₂O₃/Al heat-spreader indicating a significant reduction of the sidewall reflectivity.

It has been reported that one may expect such reduction of the sidewall reflectivity for slanted mesa devices owed to the roughness induced optical losses of the metallic sidewall reflector.³⁰ In that report, the authors demonstrated that it can be cleverly circumvented by using a narrow grid geometry interconnect, although the thermal consequences were not studied. In this work, the reduced sidewall reflectivity caused by the Al₂O₃/Al heat-spreader was eventually overcome by the marked improvement of the device thermal management. This translated to an increased peak LOP in all cases. Highlighting the overarching criticality of minimizing self-heating effects and the lateral travel length of DUV photons for AlGaN LEDs, the 5 µm pixel presented here had a peak brightness and current of 570 W cm² (4 mA) DC drive when equipped with the heat-spreader as compared to 488 W cm² (3 mA) without the boost to thermal management.

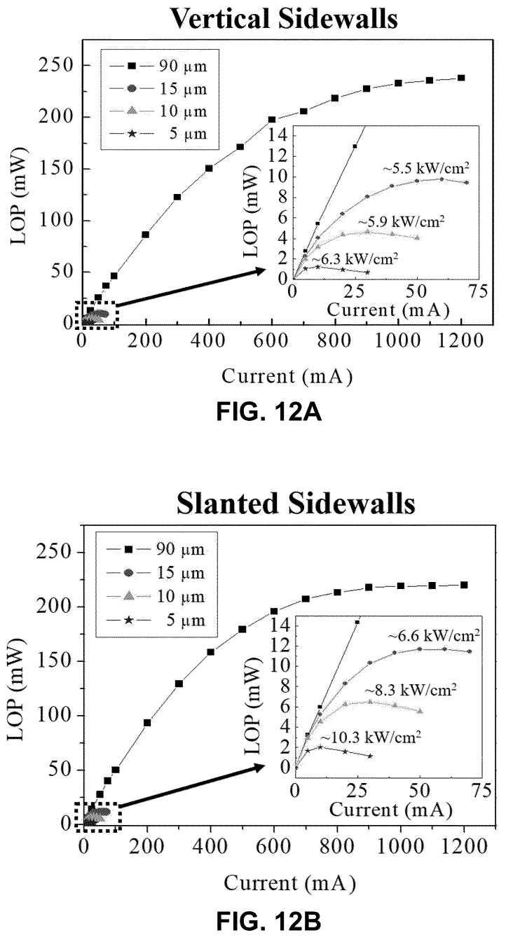

As an exemplary demonstration of the potential of sub-20 µm AlGaN DUV micro-LEDs, we also tested our heat-spreader equipped micropixels in the pulsed mode to further reduce the self-heating effect. The testing was conducted using a 500 ns pulse width and 0.05% duty cycle (FIG. 12). Under these conditions, the Kw cm² class emission brightness of our devices surpasses that of DC biased highly emissive visible micro-LEDs by at least one order of magnitude,^{4,5,36,37,38} a potentially revolutionary advance arising from the robustness of deeply scaled AlGaN micro-LEDs to ultrahigh injection current densities. We believe this level of performance can be attained in DC operation with focused innovations for the nascent sub-20 µm DUV micropixel technology that aim to simultaneously reduce the device thermal impedance and the efficiency penalty of thermal effects, lower the series resistance, increase the lateral absorption length, and improve the sidewall reflectivity.

We studied the size-dependent LEE enhancement for truncated cone AlGaN micropixel DUV LEDs with pixel sizes of 5, 10, 15 and 90 µm compared to same sized vertical-walled devices. From CL measurements, the lateral absorption length of ~15 µm of our mesa structures was determined, which is significantly shorter than for DUV waveguiding in the Al_{0.65}Ga_{0.35}N cladding layers. This indicates strong re-absorption of sideways travelling TM-polarized DUV photons by the MQW and p-GaN epitaxial layers. In direct agreement with the CL measurement, from I-V-L testing we found the LEE enhancement to follow a 1/r dependence on the mesa perimeter-to-area ratio. Hence, for DUV emitters, scaling down to sub-20 µm device dimensions, is critical for maximizing LEE. Unlike visible emission micro-LEDs, our AlGaN-based micro-LEDs do not show pronounced edge re-combination effects at such deeply scaled dimensions. The peak LOP improved further after the devices were equipped with a semi-reflective Al₂O₃/Al heat-spreader owed to an improved thermal management, despite the additional optical losses. The output power of a 5 µm diameter LED exceeded 2 mW (10.3 kW cm²) at 10 mA (50.1 kA cm²) with 500 ns pulsed current injection at 0.05% duty cycle, which emphasizes the potential of the AlGaN micropixel technology to revolutionize optical communication and lighting systems requiring emission in the DUV.

This written description uses examples to disclose the presently disclosed subject matter, including the best mode, and also to enable any person skilled in the art to practice the presently disclosed subject matter, including making and using any devices or systems and performing any incorporated methods. The patentable scope of the presently disclosed subject matter is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal language of the claims.

### REFERENCES

¹ S. Nakamura, T. Mukai, and M. Senoh, Appl. Phys. Lett. 64, 1687 (1994)
² R.X. Ferreira, E. Xie, J.J. McKendry, S. Rajbhandari, H. Chun, G. Faulkner, S. Watson, A.E. Kelly, E. Gu, R.V. Penty, and I.H. White, IEEE Phot. Techn. Lett. 28, 2023 (2016).
³ P. Tian, X. Liu , S. Yi , Y. Huang , S. Zhang , X. Zhou , L. Hu, L. Zheng, and R. Liu, Opt. Expr. 25, 1193 (2017).
⁴ M.D. Dawson, J. Hermsdorf, E. Xie, E. Gu, J. McKendry, A.D. Griffiths, and M.J. Strain, Imaging 10, 40 (2015).
⁵ Z. Chen, S. Yan, and C. Danesh, J. Phys. D: Appl. Phys. 54, 123001 (2020).
⁶ J.Y. Lin, and H.X. Jiang, Appl. Phys. Lett. 116, 100502 (2020).
⁷ S. Nagai, K. Yamada, A. Hirano, M. Ippommatsu, M. Ito, N. Morishima, K. Aosaki, Y. Honda, H. Amano, and I. Akasaki, J. Journ. Appl. Phys. 55, 082101 (2016).
⁸ V. Kramar, IEEE Proc. 27th Conf. Open Innovations Association (FRUCT), 2020, p. 90.
⁹ G.Y. Lui, D. Roser, R. Corkish N.J. Ashbolt, and R. Stuetz, Sci. Tot. Env. 553, 626 (2016).
¹⁰ M. Kneissl, T.Y. Seong, J. Han, and H. Amano, Nat. Photonics. 13, 233 (2019).
¹¹ S. Khan, D. Newport, and S. Le Calvé, Sensors. 19, 5210 (2019).
¹² Z. Xu, and B.M. Sadler, IEEE Comms. Mag. 46, 67 (2008).
¹³ H.Y. Ryu, I.G. Choi, H.S. Choi, and J.I. Shim, Appl. Phys. Expr. 6, 062101 (2013).
¹⁴W. Liu, and A.A. Balandin, J. Appl. Phys. 97, 073710 (2005).
¹⁵ M.R. Krames, O.B. Shchekin, R. Mueller-Mach, G.O. Mueller, L. Zhou, G. Harbers, and M.G. Craford, J. Disp. Technol. 3, 160 (2007).
¹⁶ J.W. Lee, D.Y. Kim, J.H. Park, E.F. Schubert, J. Kim, J. Lee, Y.I. Kim, Y. Park, and J.K. Kim, Sci. Rep. 6, 1 (2016).
¹⁷ R. Floyd, K. Hussain, A. Mamun, M. Gaevski, G. Simin, M.V.S. Chandrashekhar, and A. Khan, Appl. Phys. Expr. 13, 022003 (2020).
¹⁸ M. Shatalov, G. Simin, V. Adivarahan, A. Chitnis, S. Wu, R. Pachipulusu, V. Mandavilli, K. Simin, J.P. Zhang, J.W. Yang, and M.A. Khan, J. Journ. Appl. Phys. 41, 5083 (2002).
¹⁹ V. Adivarahan, S. Wu, W.H. Sun, V. Mandavilli, M.S. Shatalov, G. Simin, J.W. Yang, H.P. Maruska, and M.A. Khan, Appl. Phys. Lett. 85, 1838 (2004).
²⁰ M. Shatalov, Z. Gong, M. Gaevski, S. Wu, W. Sun, V. Adivarahan, and M.A. Khan, Int. Soc. Opt. Phot., 2006, 6134, p. 61340P.
²¹ R. Floyd, M. Gaevski, M.D. Alam, S. Islam, K. Hussain, A. Mamun, S. Mollah, G. Simin, M.V.S. Chandrashekhar, and A. Khan, Appl. Phys. Expr. 14, 014002 (2020).
²² S. Wu, S. Chhajed, L. Yan, W. Sun, M. Shatalov, V. Adivarahan, and M.A. Khan, Jpn. J. Appl. Phys. 45, L352 (2006).
²³ M.E. Gaevski, M. Shatalov, S. Wu, and M.A. Khan, MRS Proc. Lib. 916, 9 (2006).
²⁴ X. He, E. Xie, M.S. Islim, A.A. Purwita, J.J. McKendry, E. Gu, H. Haas, and M.D. Dawson, Phot. Res. 7, B41 (2019).
²⁵ D.M. Maclure, J.J. McKendry, J. Hermsdorf, X. He, E. Xie, E. Gu, and M.D. Dawson, 2020 IEEE Phot. Conf. (IPC), 2020, p. 1.
²⁶ J.J. McKendry, D. Massoubre, S. Zhang, B.R. Rae, R.P. Green, E. Gu, R.K. Henderson, A.E. Kelly, and M.D. Dawson, J. Light. Tech. 30, 61 (2011).
²⁷ A. Rashidi, M. Monavarian, A. Aragon, A. Rishinaramangalam, and D. Feezell, IEEE Elec. Dev. Lett. 39, 520 (2018).
²⁸ J.W. Lee, J.H. Park, D.Y. Kim, E.F. Schubert, J. Kim, J. Lee, Y.L. Kim, Y. Park, and J.K. Kim, ACS Phot. 3, 2030 (2016).
²⁹ Q. Chen, H. Zhang, J. Dai, S. Zhang, S. Wang, J. He, R. Liang, Z.H. Zhang, and C. Chen, IEEE Phot. Journ. 10, 1 (2018).
³⁰ Zhang, J., Chang, L., Zheng, Y., Chu, C., Tian, K., Fan, C., Zhang, Y. and Zhang, Z.H., Opt. Expr. 28, 17035 (2020).
³¹ Y. Zheng, J. Zhang, L. Chang, C. Chu, K. Tian, Q. Zheng, Q. Li, Y. Zhang, W. Bi, and Z.H. Zhang, J. Appl. Phys. 128, 093106 (2020).
³² One of our AIN papers
³³ Z. Zhang, M. Kushimoto, T. Sakai, N. Sugiyama, L.J. Schowalter, C. Sasaoka, and H. Amano, J. Journ. Appl. Phys. 59, 094001 (2020).
³⁴ Z. Liu, K. Wang, X. Luo, and S. Liu, Opt. Expr. 18, 9398 (2010).
³⁵ G.D. Hao, N. Tamari, T. Obata, T. Kinoshita, and S.I. Inoue, Opt. Expr. 25, A639 (2017).
³⁶ J. Hermsdorf, J.J. McKendry, S. Zhang, E. Xie, R. Ferreira, D. Massoubre, A.M. Zuhdi, R.K. Henderson, I. Underwood, S. Watson, and A.E. Kelly, IEEE Trans. Elec. Dev. 62, 1918 (2015).
³⁷ F. Templier, J. Soc. Inform. Disp. 24, 669 (2016).
³⁸ L. Dupré, M. Marra, V. Verney, B. Aventurier, F. Henry, F. Olivier, S. Tirano, A. Daami, and F. Templier, Gall. Nitr. Mats. Dev. XII. 10104, 1010422 (2017).

What is claimed is:

1. A light-emitting diode (LED), comprising an AlGaN-based micropixel LED device having a pixel p-contact diameter size of 20 µm or less, and operating in the deep ultraviolet (DUV) spectral region having wavelength emissions of less than 300 nm.

2. An LED as in claim 1, wherein said LED device further includes an added heat sink layer for efficient DUV light production at increased input power levels.

3. An LED as in claim 2, further comprising a plurality of said LEDs individually connected together in a matrix subarray with respective pixel spacing of at least 5 µm.

4. An LED as in claim 3, further comprising a plurality of said matrix subarrays interconnected together to form an array of subarrays.

5. An LED as in claim 4, further comprising a plurality of said subarrays connected together in a matrix interconnected by an Al-based heat sink.

6. An LED as in claim 2, further comprising a plurality of said LEDs connected together in a modular array to form an LED lamp, and including a pulse mode ultrahigh injection current density power source for powering said LED lamp.

7. An LED as in claim 6, wherein said power source uses a 500 ns pulse width and 0.05% duty cycle.

8. An LED as in claim 1, wherein said LED device comprises a truncated cone AlGaN DUV micropixel LED with pixel size in a range from 20 to 5 µm.

9. An LED as in claim 8, wherein said LED device further includes an added semireflective Al₂O₃/Al heat spreader layer to act as a heat sink.

10. An LED as in claim 1, further comprising a plurality of said LEDs connected together in a modular array by a metal heat sink.

11. An LED as in claim 10, wherein p-metal dimensions for the respective pixels are one of 5, 10 and 15 µm diameter, and said respective pixels have spacing of at least 5 µm.

12. An LED as in claim 1, further comprising a plurality of said LEDs interconnected with the n-contact network blanket removed between individual LEDs so as to form a border of n-contact features around the interconnected LEDs.

13. An LED as in claim 12, wherein said plurality of LEDs have respective pixel mesa sidewalls which are respectively inclined or vertical.

14. An LED as in claim 13, wherein said plurality of LEDs have respective pixel mesa sidewalls which are respectively slanted at angles of 48 degrees or less.

15. An LED as in claim 12, further wherein said plurality of said LEDs are connected to a common supply terminal.

16. An LED as in claim 12, wherein said interconnected LEDs include a layer of reflective aluminum heat spreader material to interconnect individual pixels of said LEDs.

17. An LED as in claim 1, wherein said LED device comprises a truncated cone AlGaN DUV micropixel LED with pixel structure comprising a mesa with slanted sidewalls, wherein the ratio of the sidewall surface area to the mesa volume is at least 0.2.

18. A modular LED array, comprising:
a plurality of respective aluminum gallium nitride (AlGaN) multiple quantum well (MQW) micropixel light-emitting diodes (LEDs) operating in the deep ultraviolet (DUV) spectral region with λₑₘᵢₛₛᵢₒₙ < 300 nm; and
said plurality of AlGaN MQW DUV LEDs respectively arranged in an array interconnected by a metal heat sink, and connected to a common supply terminal;
wherein said LEDs have respective pixel sizes from 5 to 20 µm in diameter, and respectively have an added heat sink layer.

19. A modular LED array as in claim 18, wherein said heat sink layer for each respective LED comprises a respective layer of Al-based heat spreader material.

20. A modular LED array as in claim 18, wherein said LEDs are connected with a common supply terminal.

21. A modular LED array as in claim 20, wherein said modular LED array comprises a lighting system further comprising a pulse mode ultra-high injection current density power source connected to said common supply terminal.

22. A modular LED array as in claim 18, further comprising a plurality of said modular LED arrays interconnected together.

23. A modular LED array as in claim 22, further combined with electroplating and flip chip packaging.

24. A modular LED array as in claim 18, wherein said LEDs respectively comprise a truncated cone AlGaN DUV micropixel LED with pixel structure comprising a mesa with slanted sidewalls, wherein the ratio of the sidewall surface area to the mesa volume is at least 0.2.

25. A modular LED array as in claim 24, wherein said plurality of LEDs have respective pixel mesa sidewalls which are respectively slanted at angles of 48 degrees or less.

26. A modular LED array as in claim 18, wherein said respective pixels have spacing of at least 5 µm.

27. Methodology for forming a light-emitting diode (LED) modular device, comprising:
fabricating an AlGaN-based micropixel LED device operable in the deep ultraviolet (DUV) spectral region as to have a pixel diameter size of 20 µm or less.

28. Methodology as in claim 27, further comprising adding a heat sink layer to said micropixel LED device for efficient DUV light production at increased input power levels.

29. Methodology as in claim 28, wherein said LED device comprises a truncated cone AlGaN DUV micropixel LED with pixel size in a range from 20 to 5 µm.

30. Methodology as in claim 29, wherein:
said plurality of LEDs have respective pixel mesa sidewalls which are respectively inclined or vertical; and
the ratio of the sidewall surface area to the mesa volume is at least 0.2.

31. Methodology as in claim 30, wherein said plurality of LEDs have respective pixel mesa sidewalls which are respectively slanted at angles of 48 degrees or less.

32. Methodology as in claim 28, further comprising interconnecting a plurality of said LEDs together in a modular array using a metal heat sink.

33. Methodology as in claim 32, further comprising connecting said plurality of said LEDs with a pulse mode ultra-high injection current density power source.

34. Methodology as in claim 33, further comprising operating said power source to produce 500 ns pulse width pulses at a 0.05% duty cycle.

35. Methodology as in claim 32, further comprising using DUV light production from said modular array for air purification, water purification both large scale and point-of-use, germ killing and viral deactivation applications, sterilization of surfaces, deep ultraviolet optical communications, polymer curing, sterilization of food, or for microscale light emission source, and/or detector for DUV photonics integrated circuits.

36. Methodology as in claim 28, further comprising interconnecting a plurality of said LEDs together in a matrix subarray with respective pixel spacing of at least 5 µm.

37. Methodology as in claim 36, further comprising fabricating a plurality of said matrix subarrays interconnected together to form an array of subarrays.

38. Methodology as in claim 37, further comprising fabricating a plurality of said subarrays connected together in a matrix interconnected by an Al-based heat sink.

39. Methodology as in claim 36, further comprising interconnecting said LEDs with a layer of reflective aluminum heat spreader material.

40. Methodology as in claim 28, further comprising fabricating a plurality of said LEDs interconnected with the n-contact network blanket removed between individual LEDs so as to form a border of n-contact features around the interconnected LEDs.
